(19) United States
(12) Patent Application Publication
(10) Pub. No.: US 2022/0081675 A1
(43) Pub. Date: Mar. 17, 2022

(54) METHOD FOR PREPARATION OF BLASTOCYST

(71) Applicant: KITAZATO CORPORATION, Shizuoka (JP)

(72) Inventors: Shingo MIYAMOTO, Kasuga-shi (JP); Fumitoshi KOGA, Fukuoka-shi (JP); Fusanori YOTSUMOTO, Fukuoka-shi (JP); Toyofumi HIRAKAWA, Fukuoka-shi (JP)

(21) Appl. No.: 17/475,877

(22) Filed: Sep. 15, 2021

(30) Foreign Application Priority Data
Sep. 15, 2020 (JP) 2020-154759

(51) Int. Cl. C12N 5/073; A61K 35/54

(52) U.S. Cl. CPC C12N 5/0603; C12N 2506/04; C12N 2502/1382; A61K 35/54

(57) ABSTRACT

Provided a method for preparing a blastocyst from a fertilized egg. The method for preparing a blastocyst includes a culture of a fertilized egg in a mixed culture medium containing a culture medium for stem cells and a culture medium for a fertilized egg, wherein the culture of a fertilized egg is performed together with a culture of adipose tissue-derived stem cells apart from the fertilized egg.

### CROSS-REFERENCE TO RELATED APPLICATION

This application claims priority to Japanese Patent Application No. 2020-154759, filed on Sep. 15, 2020, the content of which is hereby incorporated by reference in its entirety.

### BACKGROUND

### Technical Field

The present disclosure relates to a method for preparation of blastocyst.

### Description of the Related Art

In Japan, one in five married couples is currently diagnosed with infecundity, and the number continues to increase. One of the causes of infertility is implantation failure, which is difficult to treat with the current reproductive medicine technology. For treating implantation failure, for example, regeneration of uterine endometrium, and improvement of the egg quality of a fertilized egg to be implanted are conceivable. In particular, if it is possible to activate maturation of a fertilized egg, such as differentiation induction from an embryo cell to a blastocyst, it can be expected that the implantation rate and therefore the pregnancy rate will be significantly improved.

For example, in Japanese Patent Application Laid-Open No.2016-007161, it is described that an egg is activated when adipose tissue-derived stem cells and the egg are co-cultured in a mutually contact state. Further, it is described in Molecular Reproduction & Development 80: 1035-1047, 2013 that a physiologically active substance secreted from human adipose tissue-derived mesenchymal stem cells improves the growth of a porcine embryo.

### SUMMARY

The first aspect is a method for preparing a blastocyst including a culture of a fertilized egg in a mixed culture medium containing a culture medium for stem cells and a culture medium for a fertilized egg, wherein the culture of a fertilized egg is performed together with a culture of adipose tissue-derived stem cells apart from the fertilized egg. In the culture of a fertilized egg by the preparation method, the fertilized egg and the adipose tissue-derived stem cells may be co-cultured in a mixed culture medium in the same vessel, or the culture of the adipose tissue-derived stem cells may be performed in a culture medium for adipose tissue-derived stem cells separated from the mixed culture medium for culturing the fertilized egg in a liquid-communicable state, while culturing the fertilized egg in the mixed culture medium.

In the preparation method, the biological species of the adipose tissue-derived stem cells and the biological species of the fertilized egg may be the same, or the fertilized egg may be a human fertilized egg, or a fertilized egg of a non-human mammal.

The second aspect is a method for inducing a fertilized egg to a blastocyst including a culture of a fertilized egg in a mixed culture medium containing a culture medium for stem cells and a culture medium for a fertilized egg, wherein the culture of a fertilized egg is performed together with a culture of adipose tissue-derived stem cells apart from the fertilized egg. In the culture of a fertilized egg by the induction method, the fertilized egg and the adipose tissue-derived stem cells may be co-cultured in a mixed culture medium in the same vessel, or the culture of the adipose tissue-derived stem cells may be performed in a culture medium for stem cells separated from the mixed culture medium in a liquid-communicable state.

In the induction method, the biological species of the adipose tissue-derived stem cells and the biological species of the fertilized egg may be the same, or the fertilized egg may be a human fertilized egg, or a fertilized egg of a non-human mammal.

### BRIEF DESCRIPTION OF DRAWINGS

FIG. 1 is a graph showing the cell proliferation of adipose tissue-derived stem cells in a mixed culture medium.

FIG. 2 is a graph showing the differentiation of fertilized eggs in mixed culture media.

FIG. 3 is a graph showing the differentiation of fertilized eggs in mixed culture media to which a culture supernatant of adipose tissue-derived stem cells has been added.

FIG. 4 is a graph showing the differentiation of fertilized eggs when fertilized eggs and adipose tissue-derived stem cells are co-cultured in mixed culture media.

FIG. 5 illustrates graphs showing the expression states of a differentiation marker of adipose tissue-derived stem cells in mixed culture media.

FIG. 6 is a graph showing the effect of a culture medium in a multi-layer culture of adipose tissue-derived stem cells.

FIG. 7 is a graph showing the differentiation of fertilized eggs, when fertilized eggs and adipose tissue-derived stem cells are subjected to a multi-layer culture.

FIG. 8 is a graph showing the effect of culture conditions on the differentiation of fertilized eggs.

### DETAILED DESCRIPTION

The term “step” includes herein not only an independent step, but also a step which may not necessarily be clearly separated from another step, insofar as an intended function of the step can be attained. In referring herein to the content of a component in a composition, when plural substances exist corresponding to a component in the composition, the content means, unless otherwise specified, the total amount of the plural substances existing in the composition. The upper or lower limit of a numerical range described herein may be optionally replaced with any of limit values of numerical ranges presented as examples. An embodiment of the present invention will be described in detail below. However, the following embodiment is just to present an example of a method for preparing a blastocyst for the sake of embodying the technical idea of the present invention, and the present invention is not limited to the following preparation method.

### Method for Preparing Blastocyst

The method for preparing a blastocyst includes a culture step of a fertilized egg in a mixed culture medium containing a culture medium for stem cells and a culture medium for a fertilized egg. In the culture step, the culture of a fertilized egg is performed in vitro together with a culture of adipose tissue-derived stem cells in a separate state from the fertilized egg. When a fertilized egg is cultured not in direct contact with adipose tissue-derived stem cells, differentiation induction of the fertilized egg to a blastocyst can be attained with very high efficiency. In addition, the development and differentiation rates of a fertilized egg (embryo cell) can be increased

The fertilized egg is the first cell to be formed by the union of male and female gametes of an oogamous species. When a fertilized egg is cultured, it differentiates through 2-cell stage embryo, 4-cell stage embryo, 8-cell stage embryo, and mulberry embryo, and then to blastocyst. The blastocyst is further classified into early blastocyst, mid-blastocyst, and late (expanded) blastocyst. When a fertilized egg is differentiation-induced to a blastocyst and then transplanted into the uterus, the implantation rate can be improved in a fertility treatment.

A fertilized egg may be originated from any mammal. Examples of the mammal include a human, a monkey, a pig, cattle, a horse, a goat, a sheep, a dog, a cat, a mouse, a rat, a guinea pig, and a hamster. A fertilized egg may be originated from a human or a non-human mammal.

The adipose tissue-derived stem cells to be cultured with a fertilized egg may be somatic stem cells contained in the adipose tissue. The adipose tissue-derived stem cells may be stem cells obtained by a culture (including passage culture) of somatic stem cells insofar as the pluripotency is maintained. The adipose tissue-derived stem cells may be, for example, adipose tissue-derived regeneration cells (ADRCs; Adipose Derived Regeneration Cells), or adipose tissue-derived mesenchymal stem cells (AMSCs; Adipose Delivered Mesenchymal Stem Cells) obtained by culturing ADRCs. In other words, the adipose tissue-derived stem cells may be herein any of ADRCs, AMSCs, adipose-derived stem cells (ASC), adipose tissue-derived mesenchymal stem cell (AT-MSC), etc. It is preferable to include at least one kind selected from the group consisting thereof, and it is more preferable to include at least AMSCs.

The origin of adipose tissue-derived stem cells, namely the biological species, may be the same as, or different from the biological species of a fertilized egg. The biological species of adipose tissue-derived stem cells is preferably the same as the biological species of a fertilized egg, and the origin of the adipose tissue-derived stem cells and the origin of an egg that becomes a fertilized egg are preferably the same individual.

Adipose tissue-derived stem cells can be obtained by a publicly known preparation method. For example, they may be prepared by treating adipose tissues with a deflocculant, then concentrating ADRCs (regeneration cells) by a centrifugation treatment, recovering the concentrated ADRCs, and if necessary culturing the same. Examples of the deflocculant include neutral protease, collagenase, trypsin, lipase, hyaluronidase, deoxyribonuclease, and pepsin. It is preferable to include collagenase. For details of the method for recovering regeneration cells from adipose tissues, refer, for example, to Japanese Patent Application Laid-Open No. 2012-51923, Japanese Patent Application Laid-Open No. 2012-75439, International Publication No. WO 2015/042182, International Publication No. WO 2006/127007, etc. ADRCs may also be prepared using a commercially available device (e.g., Celution System (Cytori Therapeutics Inc., San Diego, USA)).

The adipose tissue from which ADRCs are obtained may be present in any region of a living body. It is preferably a subcutaneous adipose tissue present in subcutaneous tissues. A subcutaneous adipose tissue may be obtained from any regions of a living body, such as the lumbar dorsal region, and the femoral region.

ADRCs may include at least one kind of adipose stem cells (such as AMSCs, and ASCs), progenitor cells, etc., and may further include vascular endothelial cells, vascular perithelial cells, their progenitor cells, etc. Accordingly, adipose stem cells can be purified by culturing ADRCs. ADRCs may be cultured, for example, by suspending the same in an appropriate culture medium, then seeding the suspension into a culture vessel, and incubating it overnight. The incubation conditions may be, for example, at 37° C., and 5% CO₂. Thereafter, at least a portion of the floating cells (non-adherent cells) may be removed by medium replacement. Further, the culture may be continued performing medium replacement as appropriate, e.g., once every 2 to 4 days. If necessary, a passage culture may be performed. There is no particular restriction on the passage number, and, for example, it may be limited to about three passages to about five passages. As the culture medium, an ordinary medium for culturing animal cells may be used. For example, Dulbecco's modified Eagle's culture medium (DMEM) (Nissui Pharmaceutical Co., Ltd., etc.), α-MEM (Dainippon Pharmaceutical Co., Ltd., etc.), DMEM: Ham's F12 mixed medium (1:1) (Dainippon Pharmaceutical Co., Ltd., etc.), Ham's F12 medium (Dainippon Pharmaceutical Co., Ltd., etc.), MCDB 201 medium (Research Institute for the Functional Peptides Co., Ltd), or the like may be used. Serum (such as fetal bovine serum, human serum, and ovine serum), or a serum replacement (such as knockout serum replacement (KSR)) may be added to the culture medium. The addition amount of the serum or serum replacement may be, for example, in a range of from 5% (v/v) to 30% (v/v).

When ADRCs are cultured, it is possible that adipose stem cells selectively survive and proliferate as adherent cells. Then, the proliferated adipose stem cells may be recovered. The recovery operation may employ an ordinary method, and, for example, the cells can be easily recovered by peeling the cells with a cell scraper, pipette, or the like after an enzymatic treatment (trypsin or dispase treatment). In a case where a sheet culture is performed using a commercially available temperature-sensitive culture dish or the like, it is also possible to recovery cells in a sheet form as they are, without an enzyme treatment. By using adipose stem cells recovered as above, differentiation of a fertilized egg can be inducted more effectively.

In the method for preparing a blastocyst, a culture of a fertilized egg is performed in a mixed culture medium containing a culture medium for stem cells and a culture medium for a fertilized egg. The culture medium for stem cells means a culture medium which is suitably used for culturing stem cells, and may employ an appropriate one to be selected from cell culture media ordinarily used. Examples of the culture medium for stem cells may include MEM, MEMα, DMEM, GMEM, Ham's F12 medium, IMDM, RPI 1640, D-PBS, and HBSS, and a mixed culture medium thereof is also applicable. Examples of the culture medium for a fertilized egg may include KSOM, PAM-5, ORIGIO Sequential Fert (produced by ORIGIO), ORIGIO Sequential Blast (produced by ORIGIO), ORIGIO Sequential Cleav (produced by ORIGIO), SAGE 1-Step (produced by ORIGIO), ARTEC (produced by Kojin Bio Co., Ltd.), HIGROW OVIT (produced by Fuso Pharmaceutical Industries, Ltd.), QA Cleavage Medium (produced by Sage BioPharma, Inc.), and Complete Blastocyst Medium (produced by Irvine Scientific Sales Co, Inc.) as a culture medium for a late stage, and a mixed culture medium thereof is also applicable. The mixing ratio of a culture medium for stem cells and a culture medium for a fertilized egg in the mixed culture medium may be selected as appropriate according to the culture method. The mixed culture medium may also contain additional additives such as serum, a serum replacement, plasma, serum albumin, a protein, a growth factor, cytokine, a hormone, an amino acid, a vitamins, an antibiotic, etc.

In an embodiment, the culture of a fertilized egg may be performed by a co-culture of a fertilized egg and adipose tissue-derived stem cells in a mixed culture medium in the same vessel. In this case, the co-culture is performed without direct contact between the fertilized egg and the adipose tissue-derived stem cells. When a mixed culture medium is used, differentiation of a fertilized egg can be efficiently induced while maintaining the cell number of adipose tissue-derived stem cells. The content of the culture medium for stem cells in the mixed culture medium consisting of the culture medium for stem cells and the culture medium for a fertilized egg may be, for example, 1 vol % or more, and preferably 5 vol % or more, 10 vol % or more, 15 vol % or more, 20 vol % or more, 30 vol % or more, or 40 vol % or more. The upper limit of the content of the culture medium for stem cells in the mixed culture medium may be, for example, 80 vol % or less, and preferably 60 vol % or less, 50 vol % or less, 40 vol % or less, or 30 vol % or less.

When a fertilized egg and adipose tissue-derived stem cells are co-cultured in a mixed culture medium in the same vessel, the fertilized egg and the adipose tissue-derived stem cells should be cultured respectively in separate sections in the same vessel. In addition, a partition may be provided between the section where the fertilized egg is cultured and the section where the adipose tissue-derived stem cells are cultured, or it may be omitted.

In another embodiment, the culture of a fertilized egg may be performed such that a fertilized egg is cultured in a mixed culture medium and adipose tissue-derived stem cells are cultured in a culture medium which contains a culture medium for stem cells, and is separated from the mixed culture medium in a liquid-communicable state. In this case, a fertilized egg and adipose tissue-derived stem cells may be cultured respectively in separate vessels, for example, using a first vessel for culturing a fertilized egg and a second vessel for culturing adipose tissue-derived stem cells, which is connected to the first vessel via a liquid-communicable partition. Alternatively, a fertilized egg is cultured in a first vessel containing at least a culture medium for a fertilized egg, and adipose tissue-derived stem cells are cultured in a second vessel containing a culture medium for stem cells. The first and second vessels may be arranged side by side on the same plane, or may be arranged on two different levels. From the viewpoints of differentiation of a fertilized egg and the proliferation efficiency of adipose tissue-derived stem cells, it is preferable that the first and second vessels are arranged on two different levels, and it is more preferable that the first vessel is placed on the lower level, and the second vessel is placed on the upper level.

Examples of a liquid-communicable partition dividing the first vessel from the second vessel include a permeable membrane made of a resin, such as polycarbonate, polyester, and polytetrafluoroethylene. The resin-made permeable membrane may be treated with collagen as necessary. By the collagen treatment, the cell adhesiveness is improved so that adipose tissue-derived stem cells can be cultured under a favorable condition. The thickness of a resin-made permeable membrane may be, for example, 10 μm or more and 30 μm or less. A resin-made permeable membrane can be formed by piercing permeation pores in a resin membrane. The size of the permeation pores may be, for example, 0.4 μm or more and 8 μm or less.

The culture of a fertilized egg using the first vessel and second vessel may be performed, for example, using Transwell permeable supports (manufactured by Corning Inc.).

In the first vessel, a mixed culture medium may be placed, or a culture medium for a fertilized egg may be placed. Even in a case in which a culture medium for a fertilized egg is placed in the first vessel, by pacing a culture medium for stem cells in the second vessel and connecting the two intercalating a liquid-communicable partition, the culture medium for stem cells diffuses into the first vessel to form a mixed culture medium in the first vessel. It is preferable to place a culture medium for stem cells in the second vessel. In this case adipose tissue-derived stem cells can be cultured in better condition.

The culture conditions for a fertilized egg may be generally employed conditions. For example, in the case of a human fertilized egg, they may be at 37° C., and 5% CO₂. In the case of that of a pig, cattle, horse, or the like, they may be, for example, from 38° C. to 39° C., and 5% CO₂. There is no particular restriction on the incubation time as long as a blastocyst is formed, for example, from 2 days to 10 days. The number of fertilized eggs to be cultured per each vessel may be one, or may be plural. While the number of seeded adipose tissue-derived stem cells per one fertilized egg may be, for example, from 1×10³ to 1×10⁶, preferably from 1×10³ to 1×10⁵, or from 5×10³ to 5×10⁴.

Another aspect of the present invention may be a method for inducing a fertilized egg to a blastocyst, or a method for inducing differentiation of the same, including a culture of a fertilized egg in a mixed culture medium containing a culture medium for stem cells and a culture medium for a fertilized egg, wherein the culture of a fertilized egg is performed together with a culture of adipose tissue-derived stem cells apart from the fertilized egg. Another aspect may be a method for preparing a blastocyst including a culture of a fertilized egg in a mixed culture medium containing a culture medium for stem cells and a culture medium for a fertilized egg, wherein the culture of a fertilized egg is performed together with a culture of adipose tissue-derived stem cells apart from the fertilized egg.

### Treatment Method for Infecundity

A treatment method for infecundity is a method for treating infecundity in a subject, by which a fertilized egg is cultured in a mixed culture medium containing a culture medium for stem cells and a culture medium for a fertilized egg, wherein the culture of a fertilized egg may be performed together with a culture of adipose tissue-derived stem cells apart from the fertilized egg so as to cause differentiation induction of the fertilized egg to a blastocyst, and the differentiation-induced blastocyst may be transplanted into the uterus of a subject. The implantation rate is improved by transplanting a differentiation-induced blastocyst. Any mammal may be the target animal of the treatment method for infecundity, and the mammal includes a human. The target subject may also be a non-human mammal

### Improvement Method for Implantation Failure

An improvement method for implantation failure is a method for treating implantation failure in a subject, by which a fertilized egg is cultured in a mixed culture medium containing a culture medium for stem cells and a culture medium for a fertilized egg, wherein the culture of a fertilized egg may be performed together with a culture of adipose tissue-derived stem cells apart from the fertilized egg so as to cause differentiation induction of the fertilized egg to a blastocyst, and the differentiation-induced blastocyst may be transplanted into the uterus of the subject. The implantation rate is improved by transplanting a differentiation-induced blastocyst. Any mammal may be the target animal of the improvement method for implantation failure, and the mammal includes a human. The target subject may also be a non-human mammal.

### Kit for Culturing Fertilized Egg

A kit for culturing a fertilized egg includes a culture medium for stem cells, a culture medium for a fertilized egg, adipose tissue-derived stem cells, and a culture vessel with upper and lower two layers connected intercalating a liquid-communicable membrane. The details of the culture medium for stem cells, culture medium for a fertilized egg, adipose tissue-derived stem cells, and culture vessel are as described above. A kit for culturing a fertilized egg may further include instructions for use that describe a protocol for differentiation induction from a fertilized egg to a blastocyst.

### EXAMPLES

The present invention will be more specifically described below by way of Examples, provided that the present invention is not limited to such Examples.

### Reference Example 1

### Preparation of Adipose Tissue-Derived Mesenchymal Stem Cells

A murine adipose tissue was obtained from the subcutis of both limbs of a mouse (ICR, female, 5-week-old). To 1 g to 2 g of the obtained adipose tissue, 10 mL of a 0.2% collagenase solution (GIBCO 17100-017) was added, and the adipose tissue soaked therewith was cut into thin strips with scissors. To the strips of adipose tissue, 20 mL of a 0.2% collagenase solution was added, and the mixture was shaken at 120 rpm at 37° C. for 1 hour. Then, it was filtered through a cell strainer (REF 352360 manufactured by FALCON), and the filtrate was centrifuged at 400 G for 5 min. The supernatant was removed, and the resulting pellet was dispersed in 10 mL of a phosphate-buffered saline (PBS) and centrifuged at 400 G for 5 min, which procedure was repeated three times to obtain a cell pellet. The obtained cell pellet was regarded as murine ADRCs. The murine ADRCs were cultured using MEMα/GlutaMAX (produced by Gibco) as a culture medium at 37° C. and 5% CO₂ for 5 days, and after three passages, adipose tissue-derived mesenchymal stem cells (AMSCs) were harvested. In this regard, medium replacement was performed every 3 days.

### Reference Example 2

### Culture of AMSCs in Mixed Culture Medium

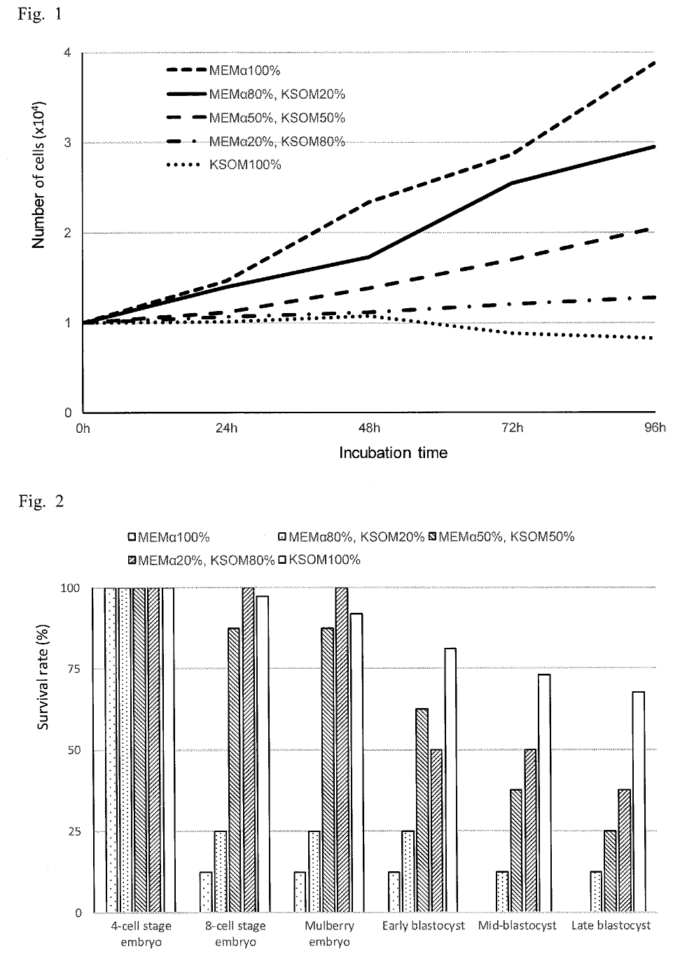

As a culture medium for stem cells MEMα/GlutaMAX (produced by Gibco, hereinafter sometimes abbreviated as MEMα) was prepared, and as a culture medium for a fertilized egg KSOM (produced by TransGenic Inc.) was prepared. The two kinds of culture media were mixed on a volumetric basis to prepare the following culture media: MEMα100%, MEMα80%+KSOM 20%, MEMα50%+KSOM 50%, MEMα 20%+KSOM 80%, and KSOM 100%. Using a Chamber Slide (8-well glass slide), 1×10⁴ AMSCs were seeded in each well, and cultured at 37° C. and 5% CO₂. The AMSCs were stained with DAPI at 24, 48, 72, and 96 hours after the start of the culture, and the cell numbers were counted. The results are shown in FIG. 1.

As shown in FIG. 1, as the mixing ratio of the culture medium for a fertilized egg increases, the cell proliferation of AMSCs is suppressed, but the effect on cell proliferation is insignificant up to the mixing ratio of the culture medium for a fertilized egg of about 20%.

### Comparative Example 1

A mixed culture medium was prepared similarly as in Reference Example 2. As fertilized eggs, 2-cell stage embryos of ICR mice were purchased, and cultured in each mixed culture medium in a 3 cm dish at 37° C. and 5% CO₂. The culture was observed every day up to 96 hours from the start of the same, and the respective survival rates up to the 4-cell stage embryo, 8-cell stage embryo, mulberry embryo, early blastocyst, mid-blastocyst, and late blastocyst were calculated. The results are shown in FIG. 2.

As shown in FIG. 2, the differentiation of the fertilized egg was suppressed as the mixing ratio of the culture medium for stem cells increased.

### Comparative Example 2

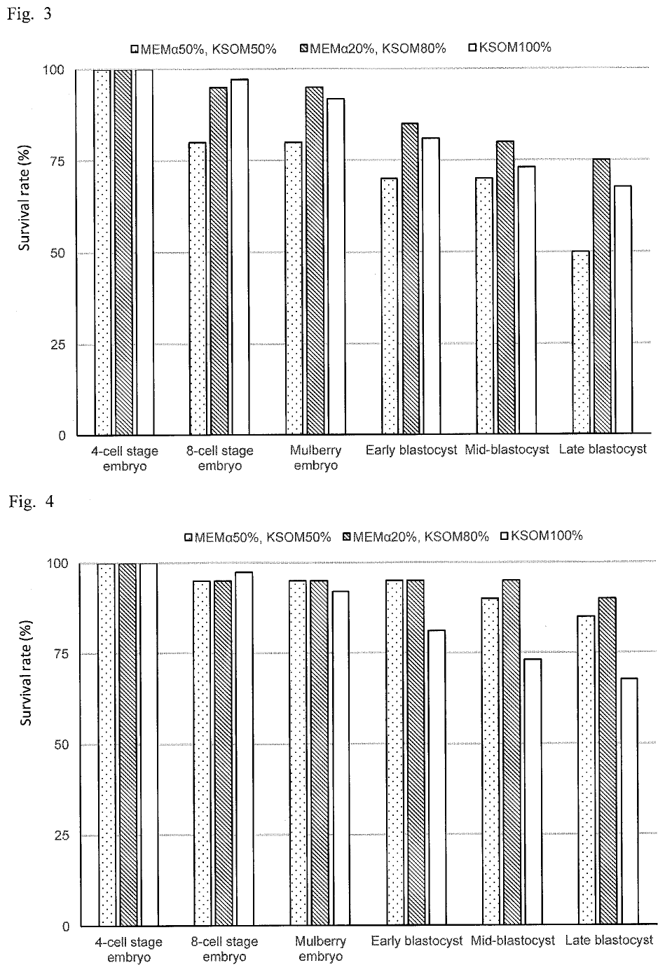

Into a 10 cm dish to which an MEMα/GlutaMAX culture medium (Gibco) was placed, 1×10⁶ AMSCs were seeded, and incubated at 37° C. and 5% CO₂ for 48 hours. After the incubation for 48 hours, the culture solution was filtrated through a 0.22 μm filter to yield a culture supernatant. The yielded culture supernatant and KSOM (TransGenic Inc.) were mixed at a ratio of 5:5 or 2:8 to prepare a mixed culture medium. Incubation of the purchased 2-cell stage embryos of ICR mice in a 3 cm dish, to which an MEMα/GlutaMAX culture medium (Gibco) was placed, was started at 37° C. and 5% CO₂. The incubation was observed every day up to 96 hours from the start of the same, and the respective survival rates up to the 4-cell stage embryo, 8-cell stage embryo, mulberry embryo, early blastocyst, mid-blastocyst, and late blastocyst were calculated. The results are shown in FIG. 3.

As shown in FIG. 3, when the culture supernatant of AMSCs was added at 20%, differentiation of a fertilized egg. was slightly promoted.

### Example 1

AS the culture media, MEMα 50%+KSOM 50%, MEMα 20%+KSOM 80%, and KSOM 100% were prepared. In a 3 cm dish to which a culture medium was placed, 1×10⁵ AMSCs and ten 2-cell stage embryos of ICR mice were seeded such that they did not come into contact with each other. The respective incubations were started simultaneously at 37° C. and 5% CO₂, and observed every day up to 96 hours from such start, and the respective survival rates up to the 4-cell stage embryo, 8-cell stage embryo, mulberry embryo, early blastocyst, mid-blastocyst, and late blastocyst were calculated. The results are shown in FIG. 4.

As shown in FIG. 4, the fertilized eggs efficiently differentiated in the mixed culture medium containing 20% of the culture medium for stem cells.

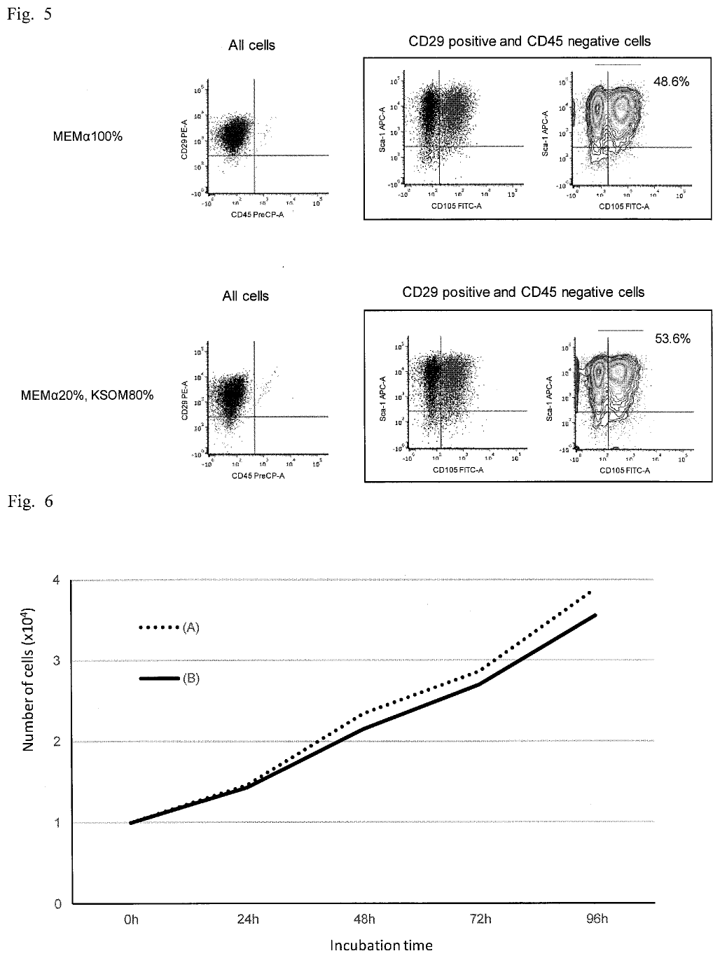

CD105, Sca-1, CD29 and CD45 as the cell surface markers of AMSCs cultured in the mixed culture medium containing 20% of the culture medium for stem cells were stained using a Mouse Mesenchymal Stem Cell Multi-Color Flow Kit (R&D Systems, Inc.), and expression of the differentiation markers on the cell surface was confirmed by flow cytometry. The results are shown in FIG. 5.

As shown in FIG. 5, the expression state of the stem cell differentiation markers in the AMSCs cultured in the mixed culture medium was similar to the expression state in the AMSCs cultured in the culture medium for stem cells.

### Reference Example 3

As a culture vessel with upper and lower two layers connected intercalating a liquid-communicable membrane, a Transwell permeable support system (manufactured by Corning Inc.; Dish diameter: 3 cm, Pore diameter: 0.4 μm) was used. MEMα/GlutaMAX (Gibco) was used as the culture medium in the upper layer, and MEMα/GlutaMAX (Gibco) or KSOM (TransGenic Inc.) was used as the culture medium in the lower layer. In the upper layer, 1×10⁵ AMSCs were seeded, and the culture was started at 37° C. and 5% CO₂. The AMSCs were stained with DAPI at 24, 48, 72, and 96 hours after the start of the culture, and the cell numbers were counted. The results are shown in FIG. 6. (A) shows the case where a culture medium for stem cells (MEMα) was used in the lower layer, and (B) shows the case where a culture medium (KSOM) for a fertilized egg was used in the lower layer.

As shown in FIG. 6, when a culture vessel with the upper and lower two layers is used, and AMSCs are cultured in the upper layer with a culture medium for stem cells, the proliferation of AMSCs is substantially not affected by the culture medium in the lower layer, whether it is a culture medium for stem cells, or a culture medium for a fertilized egg.

### Example 2

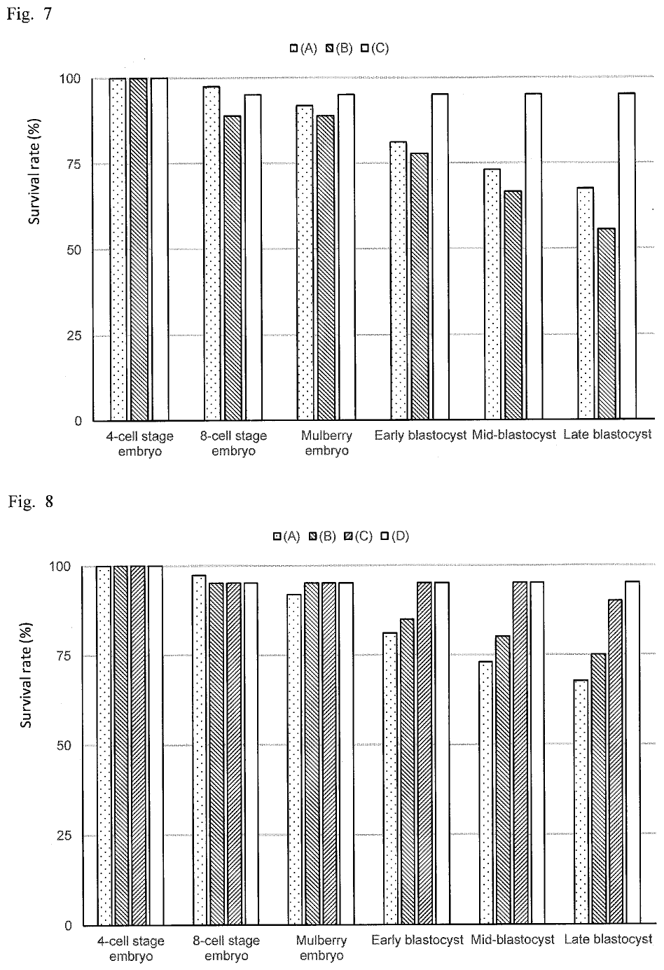

Using a Transwell permeable support system (manufactured by Corning Inc.; Dish diameter: 3 cm, Pore diameter: 0.4 μm), ten 2-cell stage embryos of ICR mice were cultured at 37° C. and 5% CO₂ in the lower layer in which a culture medium for a fertilized egg (KSOM) was placed as the culture medium. In (A), a culture medium for a fertilized egg (KSOM) was placed in the upper layer, but AMSCs were not seeded. In (B), a culture medium for stem cells (MEMα) was placed in the upper layer, but AMSCs were not seeded. In (C), a culture medium for stem cells (MEMα) was placed in the upper layer, and 1×10⁵ AMSCs were seeded. The incubation was observed every day up to 96 hours from the start thereof, and the respective survival rates up to the 4-cell stage embryo, 8-cell stage embryo, mulberry embryo, early blastocyst, mid-blastocyst, and late blastocyst were calculated. The results are shown in FIG. 7.

As shown in FIG. 7, in a case where a fertilized egg is cultured in a culture medium for a fertilized egg in the lower layer while culturing AMSCs in a culture medium for stem cells in the upper layer, the fertilized egg differentiates quite efficiently.

The effects of a culture method on differentiation of a fertilized egg in terms of the respective survival rates (%) up to the 4-cell stage embryo, 8-cell stage embryo, mulberry embryo, early blastocyst, mid-blastocyst, and late blastocyst with respect to (A) the case of a culture in a culture medium for a fertilized egg, (B) the case of a culture in a mixed culture medium to which a culture supernatant of AMSCs was added at 20% (supernatant-added culture), (C) the case of a co-culture with AMSCs in the same vessel without contact with each other in a mixed culture medium to which a culture medium for stem cells was added at 20% (co-culture without contact in the same vessel), and (D) the case where AMSCs are cultured in a culture medium for stem cells in the upper layer, and fertilized eggs are cultured in a mixed culture medium in the lower layer (multi-layer culture), are shown in Table 1 and FIG. 8.

TABLE 1

| | (A) | | (C) | |
|---|---|---|---|---|
| | Culture in | | Coculture | (D) |
| | a culture | (B) | in the same | Multi- |
| | medium for | Supernatant- | vessel with- | layer |
| | a fertilized | added culture | out contact | culture |
| | egg (%) | (%) | (%) | (%) |
| 4-cell stage | 100.0 | 100.0 | 100.0 | 100.0 |
| embryo | | | | |
| 8-cell stage | 97.3 | 95.0 | 95.0 | 95.0 |
| embryo | | | | |
| Mulberry embryo | 91.9 | 95.0 | 95.0 | 95.0 |
| Early blastocyst | 81.1 | 85.0 | 95.0 | 95.0 |
| Mid-blastocyst | 73.0 | 80.0 | 95.0 | 95.0 |
| Late blastocyst | 67.6 | 75.0 | 90.0 | 95.0 |

As shown in Table 1 and FIG. 8, when fertilized eggs are co-cultured with AMSCs without contact with each other in a mixed culture medium, the fertilized eggs differentiate efficiently.

It is to be understood that although the present invention has been described with regard to preferred embodiments thereof, various other embodiments and variants may occur to those skilled in the art, which are within the scope and spirit of the invention, and such other embodiments and variants are intended to be covered by the following claims.

Although the present disclosure has been described with reference to several exemplary embodiments, it is to be understood that the words that have been used are words of description and illustration, rather than words of limitation. Changes may be made within the purview of the appended claims, as presently stated and as amended, without departing from the scope and spirit of the disclosure in its aspects. Although the disclosure has been described with reference to particular examples, means, and embodiments, the disclosure may be not intended to be limited to the particulars disclosed; rather the disclosure extends to all functionally equivalent structures, methods, and uses such as are within the scope of the appended claims.

One or more examples or embodiments of the disclosure may be referred to herein, individually and/or collectively, by the term “disclosure” merely for convenience and without intending to voluntarily limit the scope of this application to any particular disclosure or inventive concept. Moreover, although specific examples and embodiments have been illustrated and described herein, it should be appreciated that any subsequent arrangement designed to achieve the same or similar purpose may be substituted for the specific examples or embodiments shown. This disclosure may be intended to cover any and all subsequent adaptations or variations of various examples and embodiments. Combinations of the above examples and embodiments, and other examples and embodiments not specifically described herein, will be apparent to those of skill in the art upon reviewing the description.

In addition, in the foregoing Detailed Description, various features may be grouped together or described in a single embodiment for the purpose of streamlining the disclosure. This disclosure may be not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above disclosed subject matter shall be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure may be determined by the broadest permissible interpretation of the following claims and their equivalents, and shall not be restricted or limited by the foregoing detailed description.

All publications, patent applications, and technical standards mentioned in this specification are herein incorporated by reference to the same extent as if each individual publication, patent application, or technical standard was specifically and individually indicated to be incorporated by reference.

1. A method for preparing a blastocyst comprising a culture of a fertilized egg in a mixed culture medium containing a culture medium for stem cells and a culture medium for a fertilized egg,
wherein the culture of a fertilized egg is performed together with a culture of adipose tissue-derived stem cells apart from the fertilized egg.

2. The preparation method according to claim 1, wherein the fertilized egg and the adipose tissue-derived stem cells are co-cultured in a mixed culture medium in the same vessel.

3. The preparation method according to claim 1, wherein the culture of the adipose tissue-derived stem cells is performed in a vessel which is separated from the mixed culture medium in a liquid-communicable state, and to which a culture medium for stem cells is placed.

4. The preparation method according to claim 1, wherein the biological species of the adipose tissue-derived stem cells and the biological species of the fertilized egg are identical.

5. The preparation method according to claim 1, wherein the fertilized egg is a human fertilized egg.

6. The preparation method according to claim 1, wherein the fertilized egg is a fertilized egg of a non-human mammal.

7. A method for inducing a fertilized egg to a blastocyst comprising a culture of a fertilized egg in a mixed culture medium containing a culture medium for stem cells and a culture medium for a fertilized egg,
wherein the culture of a fertilized egg is performed together with a culture of adipose tissue-derived stem cells apart from the fertilized egg.

8. A kit for culturing a fertilized egg comprising:
a culture medium for stem cells;
a culture medium for a fertilized egg;
adipose tissue-derived stem cells; and
a culture vessel with upper and lower two layers connected intercalating a liquid-communicable membrane.

9. A method for treating infecundity in a subject by which a fertilized egg is cultured in a mixed culture medium containing a culture medium for stem cells and a culture medium for a fertilized egg, wherein
the culture of a fertilized egg is performed together with a culture of adipose tissue-derived stem cells apart from the fertilized egg so as to cause differentiation induction of the fertilized egg to a blastocyst, and
the differentiation-induced blastocyst is transplanted into the uterus of a subject.

10. The treatment method according to claim 9, wherein the fertilized egg and the adipose tissue-derived stem cells are co-cultured in a mixed culture medium in the same vessel.

11. The treatment method according to claim 9, wherein the culture of the adipose tissue-derived stem cells is performed in a vessel which is separated from the mixed culture medium in a liquid-communicable state, and to which a culture medium for stem cells is placed.

12. The treatment method according to claim 9, wherein the biological species of the adipose tissue-derived stem cells and the biological species of the fertilized egg are identical.

13. The treatment method according to claim 9, wherein the fertilized egg is a human fertilized egg.

14. The treatment method according to claim 9, wherein the fertilized egg is a fertilized egg of a non-human mammal.

15. A method for treating implantation failure in a subject, by which
a fertilized egg is cultured in a mixed culture medium containing a culture medium for stem cells and a culture medium for a fertilized egg, wherein the culture of a fertilized egg is performed together with a culture of adipose tissue-derived stem cells apart from the fertilized egg so as to cause differentiation induction of the fertilized egg to a blastocyst, and
the differentiation-induced blastocyst is transplanted into the uterus of the subject.

16. The treatment method according to claim 15, wherein the fertilized egg and the adipose tissue-derived stem cells are co-cultured in a mixed culture medium in the same vessel.

17. The treatment method according to claim 15, wherein the culture of the adipose tissue-derived stem cells is performed in a vessel which is separated from the mixed culture medium in a liquid-communicable state, and to which a culture medium for stem cells is placed.

18. The treatment method according to claim 15, wherein the biological species of the adipose tissue-derived stem cells and the biological species of the fertilized egg are identical.

19. The treatment method according to claim 15, wherein the fertilized egg is a human fertilized egg.

20. The treatment method according to claim 15, wherein the fertilized egg is a fertilized egg of a non-human mammal.
